(19) United States
(12) Patent Application Publication
(10) Pub. No.: US 2003/0207947 A1
(43) Pub. Date: Nov. 6, 2003

(54) Production of lutein in microorganisms

(76) Inventors: Mervyn L. DeSouza, Plymouth, MN (US); Sherry R. Kollmann, Maple Grove, MN (US); William A. Schroeder, Brooklyn Park, MN (US)

Correspondence Address: Fish & Richardson, Suite 3300, 60 South Sixth Street, Minneapolis, MN, 55402, US

(21) Appl. No.: 10/220,948

(22) PCT Filed: Mar. 7, 2001
(86) PCT No.: PCT/US01/07178

(51) Int. Cl. C12P 23/00; C07H 21/04; C12N 9/02; C12N 9/10; C12N 1/21; A61K 31/015; C12N 1/12

(52) U.S. Cl. 514/763; 435/67; 435/69.1; 435/189; 435/193; 435/252.3; 435/252.33; 435/257.2; 536/23.2

(57) ABSTRACT

A lycopene ε cyclase from spinach is described, as is production of lutein in microorganisms by expression of the lycopene ε cyclase.

### TECHNICAL FIELD

This invention relates to production of lutein in microorganisms.

### BACKGROUND

Lutein, a carotenoid produced in many green plants, has been used to pigment animal products such as eggs, foods, drugs, and cosmetics. Shi et al., 1999, Process Biochem., 34:341-347. In addition, lutein has been used for the treatment of age-related macular degeneration, which is the leading cause of blindness in people over 55 years of age. Seddon et al., 1994, J. Am. Med. Assoc., 272:1413-1420.

Typically, lutein is produced by extraction from natural sources. For example, lutein can be extracted from marigold flowers (Tagetes erecta) with an organic solvent, then treated with an alkaline solution to provide lutein. Lutein can also be extracted from green vegetables such as kale, spinach, broccoli, green beans, and collard greens. Alternatively, algae producing lutein can be dried and used directly.

### SUMMARY

The present invention is based on the identification of a nucleic acid encoding a lycopene ε cyclase from spinach and its use for producing lutein in microorganisms that do not naturally produce or that produce small amounts of lutein. Microorganisms of the present invention can be used as nutritional supplements or as sources of lutein for food products or therapeutics purposes.

In one aspect, the invention features an isolated nucleic acid having at least 75% sequence identity to the nucleotide sequence of nucleotides 264-1817 of SEQ ID NO:5 or to a fragment of the nucleic acid of SEQ ID NO:5 at least about 34 contiguous nucleotides in length. The nucleic acid can have at least 80%, 85%, 90%, or 95% sequence identity to the nucleotide sequence of nucleotides 264-1817 of SEQ ID NO:5 or to the fragment of the nucleic acid of SEQ ID NO:5 at least about 34 contiguous nucleotides in length. An expression vector of the invention can include such a nucleic acid operably linked to an expression control element.

The invention also features an isolated nucleic acid encoding a lycopene ε cyclase polypeptide at least 75% identical to the amino acid sequence of SEQ ID NO:6 and an isolated polypeptide having at least 85% sequence identity to the amino acid sequence of SEQ ID NO:6. The isolated polypeptide can have at least 95% or 99% sequence identity to the amino acid sequence of SEQ ID NO:6.

In another aspect the invention features a microorganism that produces detectable amounts of lutein, wherein the microorganism includes an exogenous nucleic acid encoding a lycopene ε cyclase. A single polypeptide can convert α-carotene to lutein in the microorganism. The single polypeptide can be exogenous or endogenous to the microorganism. The microorganism may naturally produce β-carotene, hydroxylated derivatives of β-carotene such as zeaxanthin, or lycopene. The microorganism can be a species from a genus selected from the group consisting of Pantoea, Escherichia, and Rhodobacter. The Rhodobacter species can be R. sphaeroides or R. capsulatus. The Escherichia species can be E. vulneris. The Pantoea species can be P. stewartii. The microorganism can be a membranous bacteria.

In yet another aspect, the invention features a microorganism that includes biosynthetic enzymes (geranylgeranyl pyrophosphate synthase, phytoene synthase, phytoene dehydrogenase, lycopene β-cyclase, lycopene ε cyclase, and β-carotene hydroxylase). Each of the biosynthetic enzymes is encoded by an exogenous nucleic acid, and expression of the biosynthetic enzymes in the microorganism produces lutein. The microorganism does not naturally produce carotenoids.

A microorganism that produces detectable amounts of zeaxanthin and lutein also is featured. The microorganism includes a first exogenous nucleic acid encoding a lycopene ε cyclase operably linked to a first inducible expression control element, wherein α-carotene is converted to lutein in the microorganism by a single polypeptide, and wherein inducible expression of the first exogenous nucleic acid regulates the ratio of zeaxanthin to lutein in the microorganism. The microorganism also can include a second exogenous nucleic acid encoding a lycopene β cyclase operably linked to a second inducible expression control element, wherein inducible expression of the first and the second exogenous nucleic acids regulates the ratio of zeaxanthin to lutein in the microorganism.

The invention also features a method of producing lutein. The method includes expressing lycopene ε cyclase in a microorganism, wherein the microorganism includes an exogenous nucleic acid encoding the lycopene ε cyclase and extracting lutein from the microorganism. A single polypeptide can convert α-carotene to lutein in the microorganism.

A method of producing a feed supplement also is featured. The method includes expressing lycopene ε cyclase in a microorganism, wherein the microorganism includes an exogenous nucleic acid encoding the lycopene ε cyclase; and drying the microorganism to produce the feed supplement. A single polypeptide can convert α-carotene to lutein in the microorganism.

In yet another aspect, the invention features a method of producing an enhanced food product. The method includes expressing lycopene ε cyclase in a microorganism, wherein the microorganism comprises an exogenous nucleic acid encoding the lycopene ε cyclase; extracting lutein from the microorganism; and adding the lutein to a food product to obtain the enhanced food product.

The invention features an algal cell, wherein the algal cell includes an exogenous nucleic acid encoding a lycopene ε cyclase, and wherein the algal cell produces detectable amounts of lutein.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic that depicts the biosynthetic pathway for production of lutein and zeaxanthin.

FIG. 2 is the nucleotide sequence of the spinach lycopene ε cyclase (SEQ ID NO:5). The start (ATG) and stop codons (TAA) are bolded and underlined. The coding sequence for the complete lycopene epsilon cyclase gene is from bp 264-1817.

FIG. 3 is the amino acid sequence of the spinach lycopene ε cyclase (SEQ ID NO:6).

FIG. 4 is a chromatogram of carotenoid extracts from Escherichia vulneris. Panels A and B are lutein and zeaxanthin standards, respectively. Panel C is a chromatogram of a carotenoid extract from a negative control (E. vulneris transformed with an empty vector). Panel D is a chromatogram of a carotenoid extract from E. vulneris transformed with the spinach lycopene ε cyclase.

FIG. 5 is a chromatogram of carotenoid extracts from P. stewartii. Panel A is a chromatogram of lutein and zeaxanthin standards. Panels B and C are chromatograms of carotenoid extracts from P. stewartii cells transformed with pPRONde-LEC after induction (B) or under normal culture conditions (C). Panels D and E are chromatograms of carotenoid extracts from P. stewartii cells that were transformed with pPRONde (without insert) after induction (D) or under normal culture conditions E).

### DETAILED DESCRIPTION

Lycopene ε Cyclase Nucleic Acids

The invention features nucleic acids having at least 75% sequence identity, e.g., 80%, 85%, 90%, 95%, or 99% sequence identity, to the nucleic acid sequence of nucleotides 264-1817 of SEQ ID NO:5, or fragments of the nucleic acid of SEQ ID NO:5 that are at least about 34 nucleotides in length. Generally, percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. The total number of aligned nucleotides refers to the number of nucleotides from SEQ ID NO:5 that are being aligned. Nucleic acid sequences can be aligned by visual inspection or by using sequence alignment software. For example, the Clustal algorithm of MEGALIGN® (DNASTAR, Madison, Wis., 1997) sequence alignment software can be used. In this method, sequences are grouped into clusters by examining the distances between all pairs. Clusters are aligned as pairs, then as groups. A gap penalty of 100 and a gap length penalty of 2 are used in the alignments.

As used herein, “isolated” refers to a sequence corresponding to part or all of a nucleic acid encoding a lycopene ε cyclase polypeptide, but free of sequences that normally flank one or both sides of the wild-type nucleic acid in a plant genome. An isolated nucleic acid can be, for example, a DNA molecule, provided one of the nucleic acid sequences normally found immediately flanking that DNA molecule in a naturally-occurring genome is removed or absent Thus, an isolated nucleic acid includes, without limitation, a DNA molecule that exists as a separate molecule (e.g., a cDNA or genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules of the invention are at least about 34 nucleotides in length. For example, the nucleic acid molecule can be about 34 to 45, 40-50, 50-100, or greater than 150 nucleotides in length, e.g., 200-300, 300-500, or 500-1000 nucleotides in length. Such nucleic acids, whether protein encoding or not, can be used as probes, primers, and diagnostic reagents. In some embodiments, the isolated nucleic acid molecules encode a full-length lycopene ε cyclase. Nucleic acid molecules can be DNA or RNA, linear or circular, and in sense or antisense orientation.

Isolated nucleic acids within the scope of the invention can be obtained using any method including, without limitation, common molecular cloning and chemical nucleic acid synthesis techniques. Polymerase chain reaction (PCR) techniques can be used to produce nucleic acid molecules of the invention. PCR refers to a procedure or technique in which target nucleic acids are amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers are typically 14 to 40 nucleotides in length, but can range from 10 nucleotides to hundreds of nucleotides in length. General PCR techniques are described, for example in PCR Primer: A Laboratory Manual, Ed. by Dieffenbach, C. and Dveksler, G., Cold Spring Harbor Laboratory Press, 1995. When using RNA as a source of template, reverse transcriptase can be used to synthesize complimentary DNA (cDNA) strands.

Nucleic acids encoding lycopene ε cyclase polypeptides also can be chemically synthesized, either as a single nucleic acid molecule or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a double-stranded nucleic acid molecule per oligonucleotide pair, which then can be ligated into a vector.

Isolated nucleic acids of the invention also can be obtained by mutagenesis. For example, an isolated nucleic acid that shares identity with a sequence set forth in SEQ ID NO: 5 can be mutated using common molecular cloning techniques (e.g., site-directed mutagenesis). Possible mutations include, without limitation, deletions, insertions, and substitutions, as well as combinations of deletions, insertions, and substitutions. Alignments of nucleic acids of the invention with other known sequences encoding carotenoid enzymes can be used to identify positions to modify. For example, alignment of the nucleotide sequence of SEQ ID NO:5 with another nucleic acid encoding lycopene ε cyclase provides guidance as to which nucleotides can be substituted, which nucleotides can be deleted, and at which positions nucleotides can be inserted.

In addition, nucleic acid and amino acid databases (e.g., GenBank®) can be used to obtain an isolated nucleic acid within the scope of the invention. For example, any nucleic acid sequence having homology to a sequence set forth in SEQ ID NO: 5 or any amino acid sequence having homology to a sequence set forth in SEQ ID NO: 6 can be used as a query to search GenBank®.

Furthermore, nucleic acid hybridization techniques can be used to obtain an isolated nucleic acid within the scope of the invention. Briefly, any nucleic acid having some homology to a sequence set forth in SEQ ID NO: 5 can be used as a probe to identify a similar nucleic acid by hybridization under conditions of moderate to high stringency. Moderately stringent hybridization conditions include hybridization at about 42° C. in a hybridization solution containing 25 mM KPO₄ (pH 7.4), 5×SSC, 5× Denhart's solution, 50 μg/mL denatured, sonicated salmon sperm DNA, 50% formamide, 10% Dextran sulfate, and 1-15 ng/mL probe (about 5×10⁷, cpm/μg), and wash steps at about 50° C. with a wash solution containing 2×SSC and 0.1% SDS. For high stringency, the same hybridization conditions can be used, but washes are performed at about 65° C. with a wash solution containing 0.2×SSC and 0.1% SDS.

Once a nucleic acid is identified, the nucleic acid then can be purified, sequenced, and analyzed to determine whether it is within the scope of the invention as described herein. Hybridization can be done by Southern or Northern analysis to identify a DNA or RNA sequence, respectively, that hybridizes to a probe. The probe can be labeled with biotin, digoxygenin, an enzyme, or a radioisotope such as ³²P or ³⁵S. The DNA or RNA to be analyzed can be electrophoretically separated on an agarose or polyacrylamide gel, transferred to nitrocellulose, nylon, or other suitable membrane, and hybridized with the probe using standard techniques well known in the art. See, for example, sections 7.39-7.52 of Sambrook et al., (1989) Molecular Cloning, second edition, Cold Spring harbor Laboratory, Plainview, N.Y.

Lycopene ε Cyclase, Polypeptides

The present invention also features isolated lycopene ε cyclase polypeptides having at least 85% sequence identity, e.g., 90%Yo, 95%, or 99% sequence identity, to the amino acid sequence of SEQ ID NO:6 and fragments thereof An “isolated polypeptide” has been separated from cellular components that naturally accompany it. Typically, the polypeptide is isolated when it is at least 60% (e.g., 70%, 80%, 90%, or 95%), by weight, free from proteins and naturally-occurring organic molecules that are naturally associated with it. Percent sequence identity can be determined as described above for nucleic acid molecules. The term “polypeptide” includes any chain of amino acids, regardless of length or post-translational modification. Polypeptides that are at least 85% identical to the amino acid sequence of SEQ ID NO:6 can retain the function of lycopene ε cyclase; i.e., such polypeptides are able to catalyze the formation of s rings on carotenoids such as lycopene. In general, conservative amino acid substitutions, i.e., substitutions of similar amino acids, are tolerated without affecting protein function. Similar amino acids are those that are similar in size and/or charge properties. For example, aspartate and glutamate, and isoleucine and valine, are pairs of similar amino acids. Similarity between amino acid pairs is well known in the art and can be assessed in a number of ways. Dyahoff et al., Atlas of Protein Sequence and Structure, 1978, 5(3):345-352, is a representation of the skill of the art and provides frequency tables for amino acid substitutions that can be employed as a measure of amino acid similarity.

An isolated lycopene ε cyclase polypeptide can be obtained, for example, by extraction from a natural source (e.g., a plant cell), chemical synthesis, or by production in a host. For example, a lycopene ε cyclase polypeptide of the present invention can be produced by ligating a nucleic acid molecule encoding the polypeptide into a nucleic acid construct such as an expression vector, and transforming a bacterial or eukaryotic host cell with the expression vector. In general, nucleic acid constructs include expression control elements operably linked to a nucleic acid sequence encoding a lycopene ε cyclase polypeptide. Expression control elements do not typically encode a gene product, but instead affect the expression of the nucleic acid sequence. As used herein, “operably linked” refers to connection of the expression control elements to the nucleic acid sequence in such a way as to permit expression of the nucleic acid sequence. Expression control elements can include, for example, promoter sequences, enhancer sequences, response elements, polyadenylation sites, or inducible elements. Non-limiting examples of promoters include the puf promoter from Rhodobacter sphaeroides (GenBank Accession No. E13945), the nifHDK promoter from R. sphaeroides (GenBank Accession No. AF031817), and the fliK promoter from R. sphaeroides (GenBank Accession No. U86454).

In bacterial systems, a strain of E. coli such as BL-21 can be used. Suitable E. coli vectors include, but are not limited to, the pGEX series of vectors that produce fusion proteins with glutathione S-transferase (GST). Transformed E. coli are typically grown exponentially then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites such that the cloned target gene product can be released from the GST moiety.

In eukaryotic host cells, a number of viral-based expression systems can be utilized to express lycopene ε cyclase polypeptides. A nucleic acid encoding a lycopene ε cyclase polypeptide can be cloned into, for example, a baculoviral vector such as pBlueBac (Invitrogen, San Diego, Calif.) and then used to co-transfect insect cells such as Spodoptera frugiperda (Sf9) cells with wild-type DNA from Autographa californica multiply enveloped nuclear polyhedrosis virus (AcMNPV). Recombinant viruses producing the lycopene ε cyclase polypeptides can be identified by standard methodology. Alternatively, a nucleic acid encoding a lycopene ε cyclase polypeptide can be introduced into a SV40, retroviral, or vaccinia based viral vector and used to infect suitable host cells.

A polypeptide within the scope of the invention can be “engineered” to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

Agrobacterium-mediated transformation, electroporation and particle gun transformation can be used to transform plant cells. Illustrative examples of transformation techniques are described in U.S. Pat. No. 5,204,253 (article gun) and U.S. Pat. No. 5,188,958 (Agrobacterium). Transformation methods utilizing the Ti and Ri plasmids of Agrobacterium spp. typically use binary type vectors. Walkerpeach, C. et al., in Plant Molecular Biology Manual, S. Gelvin and R. Schilperoort, eds., Kluwer Dordrecht, C1:1-19 (1994). If cell or tissue cultures are used as the recipient tissue for transformation, plants can be regenerated from transformed cultures by techniques known to those skilled in the art.

Engineered Microorganisms

A nucleic acid encoding lycopene ε cyclase polypeptide can be expressed in microorganisms so that detectable amounts of lutein are produced. As used herein, “detectable” refers to the ability to detect lutein and esters thereof using standard analytical methodology. In general, lutein can be extracted with an organic solvent such as acetone and detected by an absorption scan from 400-500 nm in the same organic solvent. In some cases, it is desirable to back-extract with a second organic solvent, such as hexane. The maximal absorbance of lutein depends on the solvent that it is in. For example, in acetone, the maximal absorbance of lutein is at 451 nm, while maximal absorbance of zeaxanthin is at 454 nm. In hexane, the maximal absorbance of lutein and zeaxanthin is 446 nm and 450 nm, respectively.

High performance liquid chromatography coupled to mass spectrometry also can be used to detect lutein. Two reverse phase columns that are connected in series can be used with a solvent gradient of water and acetone. The first column is a C30 specialty column designed for carotenoid separation (e.g., YMCä Carotenoid S3m; 2.0×150 mm, 3 mm particle size; Waters Corporation, PN CT99S031502WT) followed by a C8 Xterraä MS column (erg., Xterraä MS C8; 2.1×250 mm, 5 mm particle size; Waters Corporation, PN 186000459).

Detectable amounts of lutein include 10 μg/g dry cell weight (dcw) and greater. For example, about 10 to 10,000 μg dew, about 100 to 60,000 μg/g dcw, about 500 to 30,000 μg/dcw, about 1000 to 20,000 μg/g dcw, or about 5,000 to 15,000 μg/g dcw. With respect to algae or other plants or organisms that produce lutein, “detectable amount of lutein” is an amount that is detectable over the endogenous level in the plant or organism.

Upon expression of lycopene ε cyclase in a microorganism, the lycopene ε cyclase converts lycopene to δ-carotene by formation of an ε-ring. Subsequently, δ-carotene is converted to α-carotene by lycopene β-cyclase. As demonstrated herein, many microorganisms surprisingly can use a single hydroxylase to convert α-carotene to lutein. Without being bound by a particular mechanism, it is thought that this hydroxylase can hydroxylate the β or ε-rings of α-carotene, the β-ring of α-cryptoxanthin, or the ε-ring of physoxanthin. See, “Key to Carotenoids”, Edited by Straub, O., Birkhäuser Verlag, 1987, p. 34-36. In plants, two hydroxylases are required, one that is specific for the β-ring and one that is specific for the ε-ring. See, for example, Pogson et al., 1996, Plant Cell, 8:1627-1639. The single hydroxylase may be endogenous (i.e., a nucleic acid or polypeptide in the naturally-occurring microorganism) or exogenous to the microorganism.

Microorganisms that are suitable for producing lutein may or may not naturally produce carotenoids, and include prokaryotic and eukaryotic microorganisms, such as bacteria, yeast, and fungi. In particular, yeast such as Phaffia rhodozyma (Xanthophyllomyces dendrorhous) and Saccharomyces cerevisiae, fungi such as Neurospora crassa, Phycomyces blakesleeanus, Blakeslea trispora, and Aspergillus sp, Archaea bacteria such as Halobacterium salinarium, and Eubacteria including Pantoea species (formerly called Erwinia) such as P. stewartii, flavobacteria species such as Xanthobacter autotrophicus and Flavobacterium multivorum, Rhodobacter species such as R. sphaeroides or R. capsulatus, and Escherichia species such as E. coli and E. vulneris can be used. Eubacteria are particularly useful. In addition, algae can be used, including Haematococcus pluvialis, Dunaliella salina, Chlorella protothecoides, and Neospongiococcum excentrum. Algae produce minor amounts of lutein.

It is noted that bacteria can be membranous or non-membranous bacteria The term “membranous bacteria” as used herein refers to any naturally-occurring, genetically modified, or environmentally modified bacteria having an intracytoplasmic membrane. An intracytoplasmic membrane can be organized in a variety of ways including, without limitation, vesicles, tubules, thylakoid-like membrane sacs, and highly organized membrane stacks. Any method can be used to analyze bacteria for the presence of intracytoplasmic membranes including, without limitation, electron microscopy, light microscopy, and density gradients. See, e.g., Chory et al., (1984) J. Bacteriol., 159:540-554; Niederman and Gibson, Isolation and Physiochemical Properties of Membranes from Purple Photosynthetic Bacteria In: The Photosynthetic Bacteria, Ed. By Roderick K. Clayton and William R. Sistrom, Plenum Press, pp. 79-118 (1978); and Lueking et al., (1978) J. Biol. Chem., 253: 451-457.

Examples of membranous bacteria that can be used include, without limitation, Purple Non-Sulfur Bacteria, including bacteria of the Rhodospirillaceae family such as those in the genus Rhodobacter (e.g., R. sphaeroides and R. capsulatus), the genus Rhodospirillum, the genus Rhodopseudomonas, the genus Rhodomicrobium, and the genus Rhodopila. The term “non-membranous bacteria” refers to any bacteria lacking intracytoplasmic membrane. Membranous bacteria can be highly membranous bacteria The term “highly membranous bacteria” as used herein refers to any bacterium having more intracytoplasmic membrane than R. sphaeroides (ATCC 17023) cells have after the R. sphaeroides (ATCC 17023) cells have been (1) cultured chemoheterotrophically under aerobic condition for four days, (2) cultured chemoheterotrophically under anaerobic for four hours, and (3) harvested. Aerobic culture conditions include culturing the cells in the dark at 30° C. in the presence of 25% oxygen. Anaerobic culture conditions include culturing the cells in the light at 30° C. in the presence of 2% oxygen. After the four hour anaerobic culturing step, the R. sphaeroides (ATCC 17023) cells are harvested by centrifugation and analyzed.

Microorganisms containing an isolated nucleic acid of the invention are not required to express the isolated nucleic acid. Isolated nucleic acids can be integrated into the genome of the microorganism or maintained in an episomal state. In other words, microorganisms can be stably or transiently transfected with an isolated nucleic acid of the invention.

Any method can be used to introduce an isolated nucleic acid into a microorganism. In fact, many methods for introducing nucleic acid into cells, whether in vivo or in vitro, are well known to those skilled in the art. For example, calcium phosphate precipitation, conjugation, electroporation, heat shock, lipofection, microinjection, and viral-mediated nucleic acid transfer are common methods that can be used to introduce nucleic acid molecules into cells. In addition, naked DNA can be delivered directly to cells in vivo as describe elsewhere (U.S. Pat. Nos. 5,580,859 and 5,589,466).

Any method can be used to identify microorganisms that contain an isolated nucleic acid within the scope of the invention. For example, PCR and nucleic acid hybridization techniques such as Northern and Southern analysis can be used in some cases, immunohistochemistry and biochemical techniques can be used to determine if a microorganism contains a particular nucleic acid by detecting the expression of a polypeptide encoded by that particular nucleic acid. For example, the polypeptide of interest can be detected with an antibody having specific binding affinity for that polypeptide, which indicates that that cell not only contains the introduced nucleic acid but also expresses the encoded polypeptide. Enzymatic activities of the polypeptide of interest also can be detected or an end product (e.g., lutein) can be detected as an indication that the microorganism contains the introduced nucleic acid and expresses the encoded polypeptide from that introduced nucleic acid.

The microorganisms described herein can contain a single copy or multiple copies (e.g., about 5, 10, 20, 35, 50, 75, 100 or 150 copies), of a particular exogenous nucleic acid. All non-naturally-occurring nucleic acids are considered an exogenous nucleic acid once introduced into the cell. The term “exogenous” as used herein with reference to a nucleic acid and a particular cell refers to any nucleic acid that does not originate from that particular cell as found in nature. Nucleic acid that is naturally-occurring also can be exogenous to a particular cell. For example, an entire operon that is isolated from a bacteria is an exogenous nucleic acid with respect to a second bacteria once that operon is introduced into the second bacteria. For example, a bacterial cell (e.g., Rhodobacter) can contain about 50 copies of an exogenous nucleic acid of the invention. In addition, the cells described herein can contain more than one particular exogenous nucleic acid. For example, a bacterial cell can contain about 50 copies of exogenous nucleic acid X as well as about 75 copies of exogenous nucleic acid Y. In these cases, each different nucleic acid can encode a different polypeptide having its own unique enzymatic activity. For example, a bacterial cell can contain two different exogenous nucleic acids such that a high level of lutein is produced. In addition, a single exogenous nucleic acid can encode one or more polypeptides.

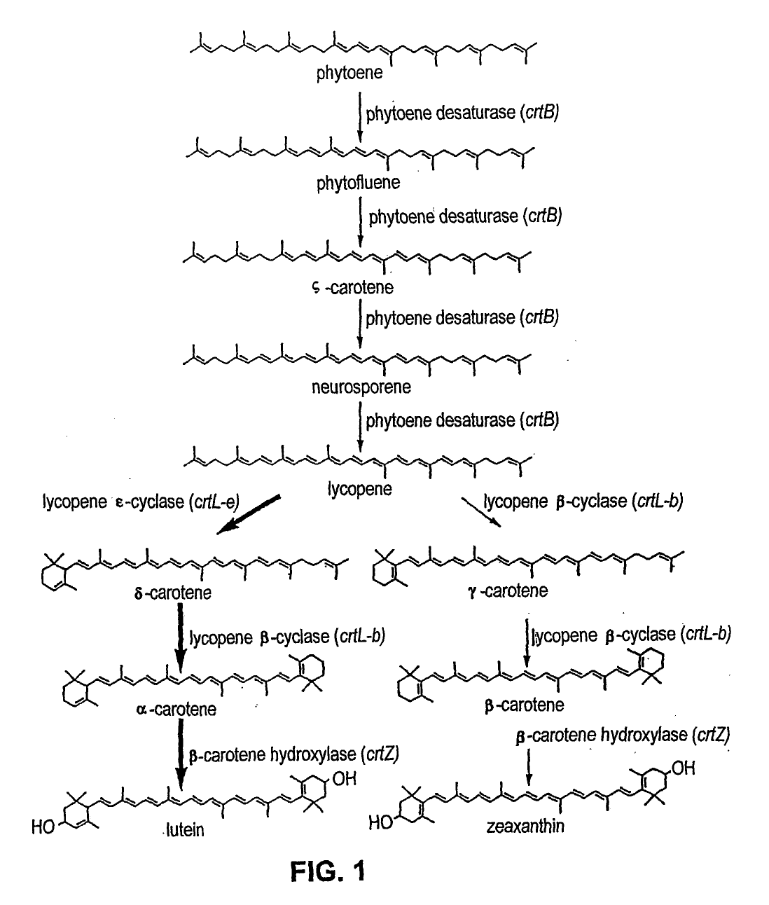

Depending on the microorganism and the metabolites present within the microorganism, one or more of the following enzymes may be expressed in the microorganism in addition to lycopene ε cyclase: geranylgeranyl pyrophosphate synthase, phytoene synthase, phytoene dehydrogenase, lycopene β cyclase, and β-carotene hydroxylase. Geranylgeranyl pyrophosphate synthase condenses one farnesyl pyrophosphate (FPP) and one isopentenyl pyrophosphate (IPP) molecule to form geranylgeranyl pyrophosphate (GGPP). Alternatively, one could utilize a multifunctional GGPP synthase that can directly prepare GGPP from IPP and dimethylallyl pyrophosphate (DMAPP). Phytoene synthase condenses two GGPP molecules to form phytoene. Phytoene dehydrogenase (also known as phytoene desaturase) performs four successive desaturations to convert phytoene to lycopene (see FIG. 1). Lycopene ε cyclase converts lycopene to δ-carotene, lycopene β cyclase converts δ-carotene to α-carotene, and β-carotene hydroxylase converts α-carotene to lutein. The genes encoding such enzymes have been cloned and sequenced from many organisms. See, for example, Genbank Accession No. Y15112 for the sequence of carotenoid biosynthesis genes of Paracoccus marousii; Genbank Accession No. D58420 for the carotenoid biosynthesis genes of Agrobacterium aurantiacum; Genbank Accession No. M87280 M99707 for the sequence of carotenoid biosynthesis genes of Erwinia herbicola; and Genbank Accession No. U62808 for carotenoid biosynthesis genes of Flavobacterium sp. Strain R1534.

For example, a microorganism that naturally produces carotenoids such as β-carotene or hydroxylated derivatives of β-carotene (e.g., zeaxanthin) can be used. Typically, β-carotene derivatives are hydroxylated on a ring and more specifically, at the 3/3′ position of the ring. Such microorganisms have the required enzymes to produce lutein after lycopene ε cyclase is expressed and is producing δ-carotene. Other suitable microorganisms naturally produce carotenoids such as lycopene, but do not produce zeaxanthin. Additional enzymes are required in these organisms for production of lutein, including lycopene β-cyclase and a hydroxylase that can convert α-carotene to lutein. In microorganisms that naturally produce FPP, but do not naturally produce carotenoids, it is necessary to introduce the enzymes that produce the substrate for lycopene ε cyclase and the enzymes to convert δ-carotene to lutein. These enzymes include geranylgeranyl pyrophosphate synthase, phytoene synthase, phytoene dehydrogenase, lycopene β cyclase, and β-carotene hydroxylase.

Control of the Ratio of Zeaxanthin to Lutein

In microorganisms that produce zeaxanthin, the ratio of zeaxanthin to lutein can be manipulated. For example, expression of lycopene β cyclase and/or lycopene ε cyclase can be controlled by an inducible promoter or by use of constitutive promoters of different strengths. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by heat, cold, salt; or toxic elements, or indirectly through the action of a pathogen or disease agent such as a virus. The inducer also can be an illumination agent such as light; darkness and light's various aspects, which include wavelength, intensity, fluorescence, direction, and duration. Examples of inducible promoters include the lac system and the tetracycline resistance system from E. coli. In one version of the Lac system, expression of lac operator-linked sequences is constitutively activated by a LacR-VP16 fusion protein and is turned off in the presence of IPTG. In another version of the Lac system, a lacR-VP16 variant is used that binds to lac operators in the presence of PTG, which can be enhanced by increasing the temperature of the cells. The lac promoter also can contain the recognition site for AraC, the repressor-inducer from the BAD promoter of the ara operon. A plasmid containing the lac promoter and an AraC recognition site (e.g., pPROLar), is available commercially from Clontech Laboratories Inc (Palo Alto, Calif.). See, Lutz and Bujard, Nucleic Acids Res. (1997) 25(6):12-3-1210.

Components of the tetracycline (Tc) resistance system also have been used to regulate gene expression. For example, the Tet repressor (TetR), which binds to tet operator sequences in the absence of tetracycline and represses gene transcription, can be used to repress transcription from a promoter containing tet operator sequences. TetR also can be fused to the activation domain of VP16 to create a tetracycline-controlled transcriptional activator (tTA), which is regulated by tetracycline in the same manner as TetR, i.e., tTA binds to tet operator sequences in the absence of tetracycline but not in the presence of tetracycline. Thus, in this system, in the continuous presence of Tc, gene expression is kept off, and to induce transcription, Tc is removed.

Alternative methods of controlling the ratio of zeaxanthin to lutein include using enzyme inhibitors to regulate the activity levels of lycopene β or ε cyclases. Examples of lycopene cyclase inhibitors include 2-(4-chlorophenylthio)-triethylamine, α-picoline, nicotine, and imidazoles. Specificity of such inhibitors for lycopene β or ε cyclases can be confirmed using extracts containing both lycopene β and ε cyclase activity.

Production of Lutein in Microorganisms

Typically, lutein is produced by culturing an engineered microorganism of the invention in culture medium and extracting lutein from the cultured microorganisms. In general, the culture media and/or culture conditions are such that the microorganisms grow to an adequate density and produce lutein efficiently. For large-scale production processes, the following methods can be used. First, a large tank (e.g., a 100 gallon, 200 gallon, 500 gallon, or more tank) containing appropriate culture medium with, for example, a glucose carbon source is inoculated with a particular microorganism. After inoculation, the microorganisms are incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank. For example, the first tank can contain medium with xylose, while the second tank contains medium with glucose. In addition, inducers can be added to the culture medium to enhance production of lutein.

Once transferred, the microorganisms can be incubated to allow for the production of lutein. Once produced, any method can be used to isolate the desired compound. For example, if the microorganism releases lutein into the broth, then common separation techniques can be used to remove the biomass from the broth, and common isolation procedures (e.g., extraction, distillation, and ion-exchange procedures) can be used to obtain lutein from the microorganism-free broth. In addition, lutein can be isolated while it is being produced, or it can be isolated from the broth after the product production phase has been terminated. If the microorganism retains lutein, the biomass can be collected and lutein can be released by treating the biomass or lutein can be extracted directly from the biomass. Once extracted, lutein can be, for example, formulated into therapeutic compositions or can be incorporated into a food product (e.g., mixed with a food product) to produce an enhanced food product.

Alternatively, the biomass can be collected and dried, without extracting the lutein. The biomass then can be formulated for human consumption (e.g., as a dietary supplement) or as an animal feed. One or more components can be added to the biomass before or after drying, including vitamins, other carotenoids, antioxidants such as ethoxyquin, vitamin E, butylated hydroxyanisole (ERA), butylated hydroxytoluene (BHT), or ascorbyl palmitate, vegetable oils such as corn oil, safflower oil, sunflower oil, or soybean oil, and an edible emulsifier, such as soy bean lecithin or sorbitan esters. Addition of antioxidants and vegetable oils can help prevent degradation of lutein during processing (e.g., drying), shipment, and storage of the composition.

The invention will be further described in the following examples, which do not limit the scope of the invention described in the claims.

### EXAMPLES

### Example 1

Cloning of Lycopene ε Cyclase from Spinach: Lycopene ε cyclase was identified from spinach (Spinacea oleracea) by amplifying a PCR product from a lambda ZAP II cDNA spinach library (Stratagene, La Jolla, Calif.). Degenerate oligonucleotides with the sequence: 5′-WAG AAA YCC YTG CCA CAT CCA TTT TG-3′, wherein W=A or T and Y=C or T (SEQ ID NO:1) and 5′-GGC CGT GCY TAT GGA AGA OTT AGT C-3′ (SEQ ID NO:1) were used to prime the PCR reaction. The nucleotide sequences of SEQ ID NOS: 1 and 2 align with nucleotides 1384-1407 and 508-532, respectively, of the open reading frame of the lycopene ε cyclase that was identified. These primers were based on homologous regions from the tomato (Lycopersicon esculentum) and Arabidopsis thaliana lycopene ε cyclase genes that did not show high homology to regions from the tomato and A. thaliana lycopene β cyclase genes. An enriched pool of products was produced by amplifying the cDNA spinach library with the degenerate primers by incubating at 96° C. for 5 minutes, followed by 25 cycles of denaturation at 96° C. for 30 seconds, annealing at 45° C. for 60 seconds, and extension at 72° C. for 2 minutes, followed by incubation at 72° C. for 10 minutes. A portion of the reaction (10 μl) was removed and reamplified with the degenerate primers (additional 25 cycles of PCR). An 899 bp PCR product was purified and sequenced from the second amplification. Non-degenerate oligonucleotides were synthesized based on the sequence of the 899 bp PCR product and used to generate an 800 bp probe to screen the cDNA library. The primers that were synthesized have the following sequences: 5′-GCA TAT GGA GCA GCT TCA GGT A-3′ (SEQ ID NO:3, nucleotides 951-972 of SEQ ID NO:5) and 5′-GGA GGT GCG GAA GAA TGT CCT GAT-3′ (SEQ ID NO:4, nucleotides 1611-1634 of SEQ ID NQ:5). Similar PCR reactions were used as described above, except that 30 cycles were used, annealing was performed at 57° C., and extension was performed for 90 seconds. A digoxigenin (DIG) labeling system from Roche Biosytems (Basel, Switzerland) was used to produce probe that was labeled by incorporation of DIG-11-dUTP during PCR.

The DIG labeled probe was purified and used to screen the cDNA spinach library. Hybridization conditions included DIG Easy Hyb solution (Boehringer Mannheim) and incubation at 42° C. for two hours, followed by two washes in 2×SSC, 0.1% SDS (w/v) for 5 minutes each at room temperature and two washes in 0.5×SSC, 0.1% SDS (w/v) for 15 minutes each at 68° C. with gentle agitation. Detection of probe was per manufacturer's instructions (Boehringer Mannheim). Plaques that hybridized to the probe were picked. Phage particles were eluted and re-plated to isolate individual plaques. The individual plaques were re-probed with the same DIG-labeled probe to ensure that the plaques were pure. From the purified lambda ZAP plaque, the pBLUESCRIPT CUBS) vector that was embedded in the phage DNA was excised. This excised pBS carried the cDNA of interest. The plasmid was purified and used to transform E. coli DH10B cells via electroporation. Plasmid DNA was isolated from colonies and sequenced.

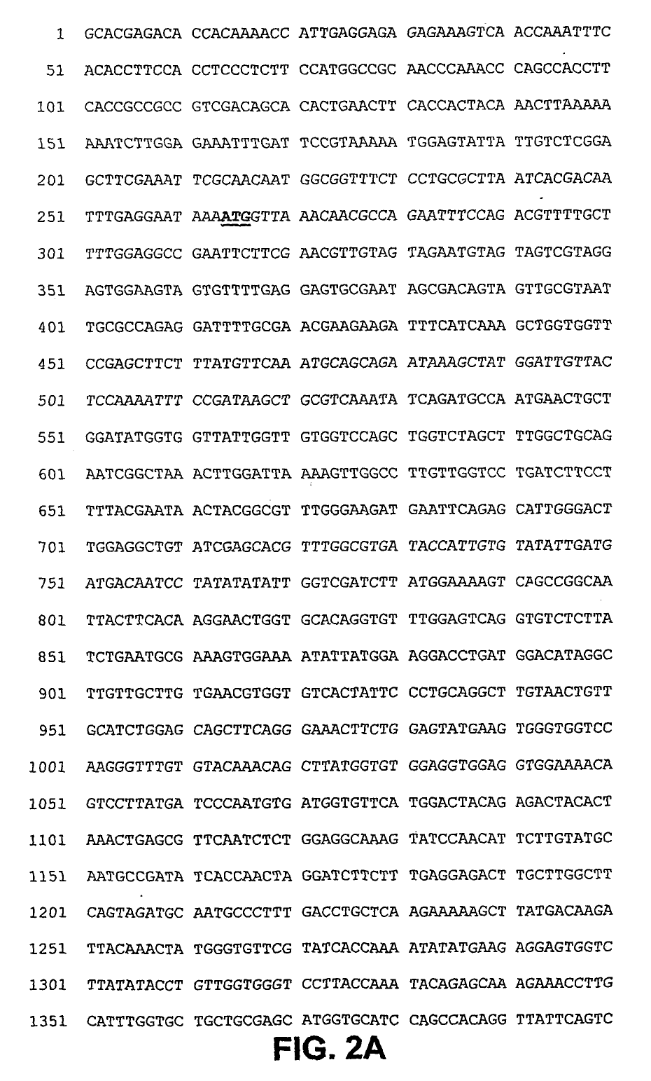

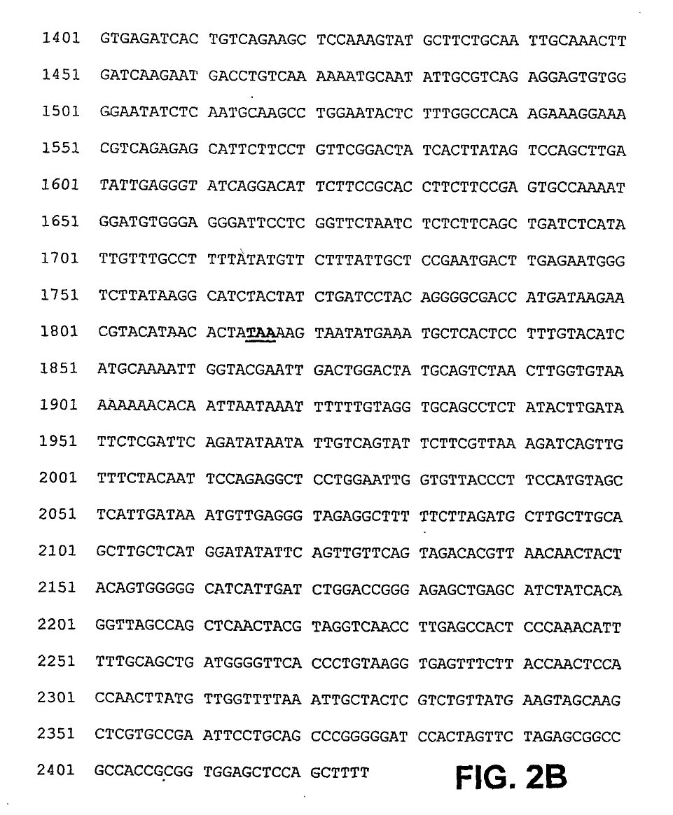

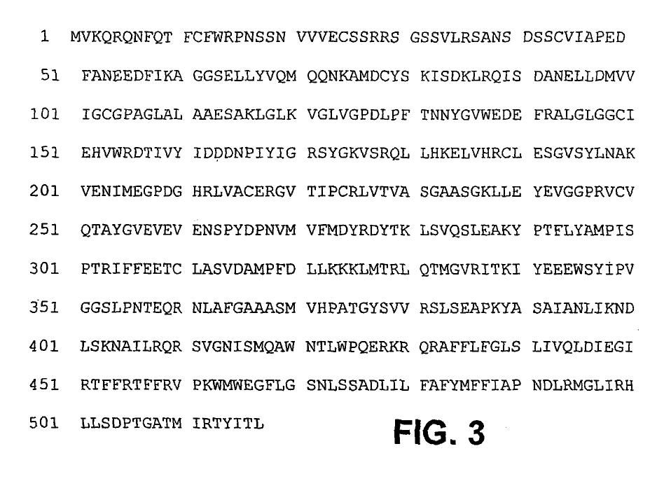

The lycopene ε cyclase cDNA has the nucleotide sequence shown in FIG. 2 (SEQ ID NO:5) and encodes a protein having the amino acid sequence shown in FIG. 3 (SEQ ID NO:6). The full-length protein is 517 amino acids in length and has a predicted molecular weight of approximately 58 kDa. The translation start site of the spinach lycopene ε cyclase gene is at nucleotide 264. The spinach lycopene ε cyclase gene has limited homology to lycopene ε cyclase genes from other plants and organisms. For example, the spinach lycopene ε cyclase gene is 66% identical to the lycopene ε cyclase from tomato, 64.6% identical to the Gentiana lutea lycopene ε cyclase, and 65.5% identical to the Arabidopsis thaliana lycopene & cyclase gene. The spinach lycopene ε cyclase has 73.2% identity to a partial sequence of the Dactus carota lycopene ε cyclase (alignment of nucleotides 664 to 1160 of spinach lycopene ε cyclase).

### Example 2

Production of Lutein in Pantoea stewartii: The spinach lycopene ε cyclase was expressed in Pantoea stewartii (formerly called Erwinia), which was obtained from the American Type Culture Collection (Accession No. 8200).

Electrocompetent P. stewartii cells were prepared by culturing a 5% inoculum of P. stewartii cells in 50 ml of LB at 26° C. with agitation (250 rpm) until an OD₅₉₀ of 0.825 was reached. The bacteria were washed in 50 ml of 10mM HEPES, pH 7.0, and centrifuged for 10 minutes at 10,000×g. The wash was repeated with 25 ml of 10 mM HEPES, pH 7.0, followed by the same centrifugation protocol. The cells then were washed once in 25 ml of 10% glycerol. Following centrifugation, the cells were resuspended in 500 μl of 10% glycerol. Aliquots were frozen and kept at −80° C. until use.

The purified pBS vector carrying the nucleic acid encoding the spinach lycopene ε cyclase (pBS:spLEC) was used to transform the P. stewartii cells by electroporation. The control was P. stewartii transformed with the pBS vector without the spinach lycopene ε cyclase cDNA. Electroporation conditions were 25 kV/cm, 200 Ohms, and 25 μfarads. Liquid cultures were grown and carotenoids were extracted by the following procedure. Approximately 20 ml cultures of P. stewartii were centrifuged at 10,000×g for 5 minutes to pellet the cells. Supernatants were decanted and the cells were washed with 10 ml of sterile, distilled water and centrifuged again. Cells were resuspended in 3 ml of acetone, scraped and crushed with a spatula, then kept in the dark for twenty minutes. After twenty minutes, the acetone extract was removed and placed in a separate tube. Approximately 1 ml of hexane was added to the tube, and the mixture was vortexed. Water (3 ml) was added to the tube and re-vortexed. The tube was centrifuged for two minutes at 1,500×g to separate the hexane and acetone/water layers. The absorbance of the hexane layer was measured in a scan from 400-500 nm. An approximate 4 nm shift in the maximum absorbance was observed between transformants expressing lycopene ε cyclase and the control cells (447 nm vs 451 nm, respectively), which is indicative of lutein production. Yield was calculated to be ˜139.5-289.411 μg lutein/g dcw.

Production of lutein was confirmed by HPLC/MS. A Waters 2690 LC System was used with two reversed-phase columns connected in series. The first column was a C30 specialty column designed for carotenoid separation (YMCä Carotenoid S3m; 2.0×150 mm, 3 mm particle size; Waters Corporation, PN CT99S031502WT) followed by a C8 Xterraä MS column (Xterraä MS C8; 2.1×250 mm, 5 mm particle size; Waters Corporation, PN 186000459). The columns are thermostated at 25° C. A Binary Gradient was used to separate lutein and zeaxanthin (Mobile Phase A: water. Mobile Phase B: acetone. Flow rate at 0.30 mL/min. Elution program: initial 90%B; 0-10 min, linear ramp up to 100%B; 10-15 min, hold at 100%B; 15-16 min, ramp down to 90%B; 16-20 min, re-equilibrate to initial conditions). Samples were dissolved in acetone prior to injection and kept cold in the autosampler (dark) at 10° C. Injection volume was 25 μl. A Photodiode Array detector, 350-550 nm, was used to detect the separated carotenoids (lutein and zeaxanthin). Under these chromatography conditions, lutein eluted at approximately 5.22 minutes and zeaxanthin eluted at approximately 5.57 minutes. Carotenoid standards were used to identify the peaks. Lutein was obtained from Sigma Chemical Co. (St. Louis, Mo.) and β-cryptoxanthin and zeaxanthin were obtained from Extrasynthese (France). UV-Vis absorption spectra were used as diagnostic features for the carotenoids as were the molecular ion and fragmentation patterns generated using mass spectrometry. A positive-ion atmospheric pressure chemical ionization mass spectrometer was used, scan range, 400-800 m/z with a quadrupole ion trap.

### Example 3

Production of Lutein in Escherichia vulneris: The spinach lycopene ε cyclase was expressed in Escherichia vulneris, which was obtained from the American Type Culture Collection (Accession No. 39368).

Electrocompetent E. vulneris cells were prepared by culturing a 1% inoculum of E. vulneris cells in 50 ml of Nutrient broth from an overnight culture. Cells were incubated 3 hours to an OD_{60D} of ˜0.68. The bacteria were washed in 50 ml of 10 mM HEPES (pH 7.0) and centrifuged for 10 minutes at 10,000×g. The wash was repeated with 25 ml of 10 mM HEPES (pH 7.0), followed by the same centrifugation protocol. The cells then were washed once in 25 ml of 10% glycerol. Following centrifugation, the cells were resuspended in 500 μl of 10% glycerol. Forty μL aliquots were frozen and kept at −80° C. until use.

Electrocompetent E. vulneris were transformed by the following electroporation procedure: 1 μL of pBS (0.072 mg, without insert) or pBS:SpLEC (0.27 μg, with spinach LEC) were added to 40 μL aliquots of electrocompetent E. vulneris and incubated on ice for 3 minutes. Cells were then electroporated at 20 kV/cm, 200 Ohms, 25 μfarads, resuspended in 1 ml SOC, and incubated at 30° C. with shaking for 60 minutes. Cells were pelleted at 10,000×g for 30 seconds and resuspended in 100 μL of fresh SOC. Aliquots of cells (75 μL and 25 μL) were plated onto separate LB+100 μg/ml Ampicillin plates and allowed to grow overnight at 26° C.

Carotenoid pigments were extracted from E. vulneris in the following manner. Single colonies of either E. vulneris transformed with pBS or E. vulneris transformed with pBS:spLEC were inoculated into 20 ml of Nutrient Broth+100 μg/ml Ampicillin. Cultures were incubated at 30° C. with shaking (250 rpm) for 5 days and harvested by centrifugation at 10,000×g for 10 min at 4° C. Cell pellets were washed once with 20 ml H₂O and resuspended in 2 ml methanol. The re-suspended cell pellets were incubated in methanol for 60 minutes at 65° C. with occasional vortexing.

Cells were pelleted by centrifugation at 10,000×g for 10 minutes at 4° C. and the methanol supernatant was removed to fresh 1.5 ml eppendorf tubes. The extracts were vacuum dried at 20° C. in a Speed Vac ISS 110 (Savant Corporation, Holbrook, N.Y.) and resuspended in a 85% methanol/15% tert-butyl methyl ether mixture.

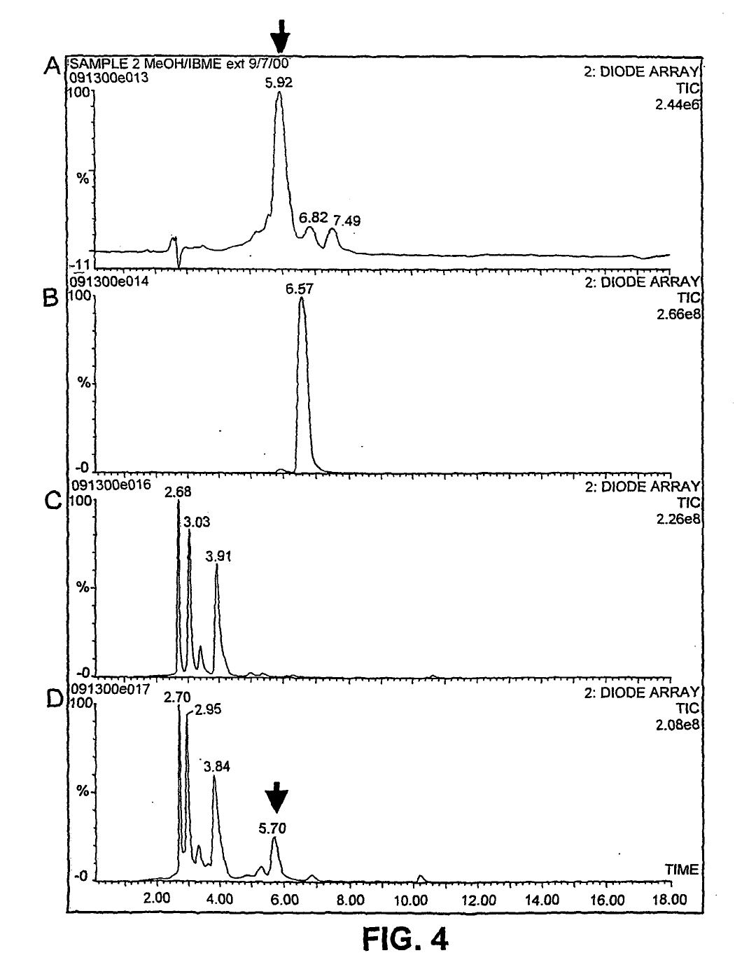

E. vulneris carotenoid extracts were analyzed by HPLC/MS as described above, except Mobile Phase A was 85% methanol/15% tert-butyl methyl ether and Mobile Phase B was 80% methanol/20% tert-butyl methyl ether. Under these chromatography conditions, lutein eluted at approximately 5.7 minutes and zeaxanthin eluted at approximately 6.6 minutes. FIG. 4 contains a representative chromatogram of carotenoid extracts from E. vulneris. Panels A and B of FIG. 4 are chromatograms of lutein and zeaxanthin standards, respectively, while panel C is a chromatogram of a carotenoid extract from a negative control (E. vulneris transformed with pBS without insert). Production of lutein in E. vulneris cells transformed with pBS:spLEC is shown in panel D of FIG. 4. The lutein peak is marked with an arrow in panels A and D. Mass spectral analysis confirmed production of lutein in E. vulneris cells transformed with pBS:spLEC.

### Example 4

Control of ratio of lutein and zeaxanthin in Pantoea stewartii: Constructs for regulating the expression of the spinach lycopene ε cyclase were produced from the pPROLarA.122 vector (ClonTech Laboratories, Inc. Palo Alto, Calif.), which includes a lac promoter that contains lac operator sequences and the recognition site for AraC, the repressor-inducer from the BAD promoter of the ara gene. A NdeI restriction site was introduced at bp 132 (nucleotide numbering as described by ClonTech laboratories) of pPROLarA.122 to generate pPRONde. The Stratagene QuikChange site specific mutagenesis kit (Stratagene, Inc., La Jolla, Calif.) was used to introduce the restriction site with the following mutagenic oligonucleotides (NdeI sites are underlined): 5′-GAGGAGAAAGGTACATATGGGTGAACAGAAAC-3′ (SEQ ID NO:7) and 5′-CAGTTTCTGTTCACCCATATGTACCTITCTCC-3′ (SEQ ID NO:8).

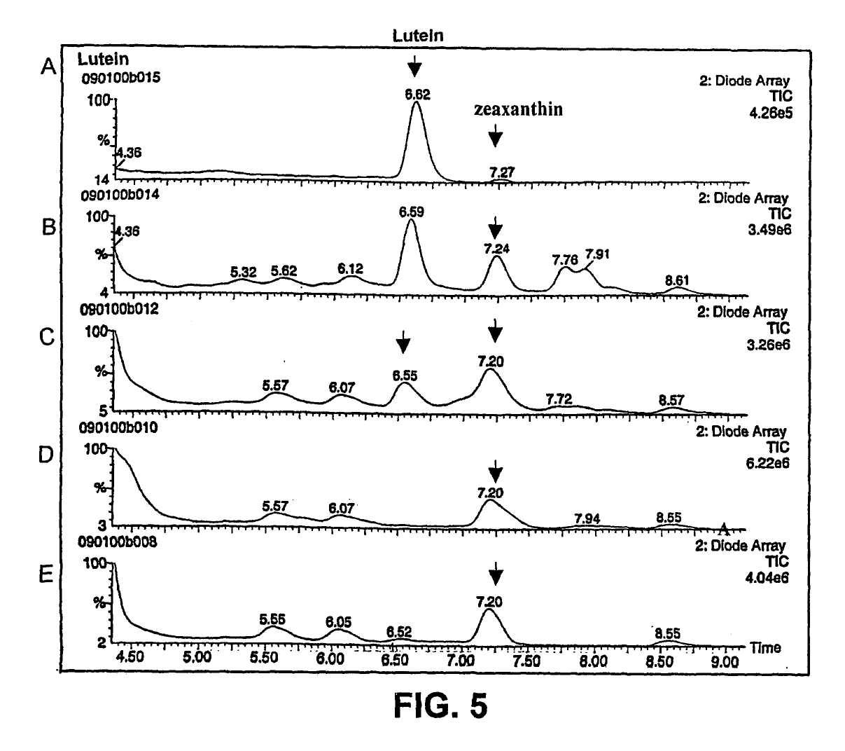

The spinach lycopene ε cyclase was cloned into the NdeI site of pPRONde to produce pPRONde-LEC, which was used to transform electrocompetent P. stewartii as described above. As a negative control, P. stewartii also were transformed with “empty” pPRONde (i.e., without the spinach lycopene ε cyclase gene). Transformed P. stewartii were cultured as described above, except that expression was induced with arabinose and IPTG in certain samples. Carotenoids were extracted and detected by HPLC analysis as described above. Lutein eluted at approximately 6.62 minutes and zeaxanthin eluted at approximately 7.27 minutes. Panel A of FIG. 5 contains chromatograms of lutein and zeaxanthin standards.

An increase in lutein production was observed in P. stewartii cells transformed with pPRONde-LEC and induced with arabinose and IPTG (Panel B of FIG. 5). There was minimal lutein production in P. stewartii cells transformed with pPRONde-LEC (Panel C of FIG. 5) and cultured under normal conditions (i.e., not induced). No lutein production was observed in P. stewartii transformed with empty vector under normal conditions or after induction with arabinose and IPTG (Panels D and E of FIG. 5, respectively).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated nucleic acid having at least 75% sequence identity to the nucleotide sequence of nucleotides 264-1817 of SEQ ID NO:5 or to a fragment of the nucleic acid of SEQ ID NO:5 at least 34 contiguous nucleotides in length.

2. The nucleic acid of claim 1, said nucleic acid having at least 80% sequence identity to the nucleotide sequence of SEQ ID NO:5 or to said fragment.

3. The nucleic acid of claim 1, said nucleic acid having at least 85% sequence identity to the nucleotide sequence of SEQ ID NO:5 or to said fragment.

4. The nucleic acid of claim 1, said nucleic acid having at least 90% sequence identity to the nucleotide sequence of SEQ ID NO:5 or to said fragment.

5. The nucleic acid of claim 1, said nucleic acid having at least 95% sequence identity to the nucleotide sequence of SEQ ID NO:5 or to said fragment.

6. An expression vector comprising the nucleic acid of claim 1 operably linked to an expression control element.

7. An isolated nucleic acid encoding a lycopene ε cyclase polypeptide at least 75% identical to the amino acid sequence of SEQ ID NO:6.

8. An isolated polypeptide having at least 85% sequence identity to the amino acid sequence of SEQ ID NO:6.

9. The isolated polypeptide of claim 8, said polypeptide having at least 95% sequence identity to the amino acid sequence of SEQ ID NO:6.

10. The isolated polypeptide of claim 8, said polypeptide having at least 99% sequence identity to the amino acid sequence of SEQ ID NO:6.

11. A microorganism, wherein said microorganism comprises an exogenous nucleic acid encoding a lycopene ε cyclase, and wherein said microorganism produces detectable amounts of lutein.

12. The microorganism of claim 11, wherein α-carotene is converted to lutein in said microorganism by a single polypeptide.

13. The microorganism of claim 11, wherein said microorganism naturally produces β-carotene or hydroxylated derivatives of β-carotene.

14. The microorganism of claim 11, wherein said microorganism naturally produces zeaxanthin.

15. The microorganism of claim 11, wherein said microorganism naturally produces lycopene.

16. The microorganism of claim 12, wherein said polypeptide is endogenous to said microorganism.

17. The microorganism of claim 12, wherein said polypeptide is exogenous to said microorganism.

18. The microorganism of claim 11, wherein said microorganism is a species from a genus selected from the group consisting of Pantoea, Escherichia, and Rhodobacter.

19. The microorganism of claim 18, wherein said Rhodobacter species is R. sphaeroides or R. capsulatus.

20. The microorganism of claim 18, wherein said Escherichia species is E. vulneris.

21. The microorganism of claim 18, wherein said Pantoea species is P. stewartii.

22. The microorganism of claim 11, wherein said microorganism is a membranous bacteria.

23. A microorganism comprising biosynthetic enzymes, wherein said biosynthetic enzymes are geranylgeranyl pyrophosphate synthase, phytoene synthase, phytoene dehydrogenase, lycopene β-cyclase, lycopene ε cyclase, and β-carotene hydroxylase, wherein each of said biosynthetic enzymes is encoded by an exogenous nucleic acid, wherein expression of said biosynthetic enzymes in said microorganism produces lutein, and wherein said microorganism does not naturally produce carotenoids.

24. A microorganism that produces detectable amounts of zeaxanthin and lutein, wherein said microorganism comprises a first exogenous nucleic acid encoding a lycopene ε cyclase operably linked to a first inducible expression control element, wherein a carotene is converted to lutein in said microorganism by a single polypeptide, and wherein inducible expression of said first exogenous nucleic acid regulates the ratio of zeaxanthin to lutein in said microorganism.

25. The microorganism of claim 24, wherein said microorganism comprises a second exogenous nucleic acid encoding a lycopene β cyclase operably linked to a second inducible expression control element, wherein inducible expression of said first and said second exogenous nucleic acids regulates the ratio of zeaxanthin to lutein in said microorganism.

26. A method of producing lutein, said method comprising expressing lycopene ε cyclase in a microorganism, wherein said microorganism comprises an exogenous nucleic acid encoding said lycopene ε cyclase; and extracting lutein from said microorganism.

27. The method of claim 26, wherein α-carotene is converted to lutein in said microorganism by a single polypeptide.

28. A method of producing a feed supplement, said method comprising expressing lycopene ε cyclase in a microorganism, wherein said microorganism comprises an exogenous nucleic acid encoding said lycopene ε cyclase; and drying said microorganism to produce said feed supplement.

29. The method of claim 28, wherein α-carotene is converted to lutein in said microorganism by a single polypeptide.

30. A method of producing an enhanced food product, said method comprising expressing lycopene ε cyclase in a microorganism, wherein said microorganism comprises an exogenous nucleic acid encoding said lycopene ε cyclase; extracting lutein from said microorganism; and adding said lutein to a food product to obtain said enhanced food product.

31. An algal cell, wherein said algal cell comprises an exogenous nucleic acid encoding a lycopene ε cyclase, and wherein said algal cell produces detectable amounts of lutein.
